(19) United States
(12) Patent Application Publication
(10) Pub. No.: US 2002/0002294 A1
(43) Pub. Date: Jan. 3, 2002

(54) Estrogenic compounds as antiangiogenic agents

(76) Inventors: Robert J. D' Amato, Cambridge, MA (US); Ravi K. Varma, Rockville, MD (US); Rudiger G. Haugwitz, Bethesda, MD (US); Mark Cushman, West Lafayette, IN (US)

Correspondence Address: KILPATRICK STOCKTON LLP, Attn: John S. Pratt, Suite 2800, 1100 Peachtree Street, Atlanta, GA, 30309-4530, US

(21) Appl. No.: 09/899,702

(22) Filed: Jul. 5, 2001

Related U.S. Application Data
(63) Continuation of application No. 09/154,322, filed on Sep. 16, 1998, now abandoned.
(60) Provisional application No. 60/059,916, filed on Sep. 24, 1997.

(51) Int. Cl. C07J 9/00; C07J 41/00

(52) U.S. Cl. 552/9; 552/515; 552/553; 552/554

(57) ABSTRACT

Compositions and methods for treating mammalian disease characterized by undesirable angiogenesis by administering derivatives of 2-methoxyestradiol of the general formula:

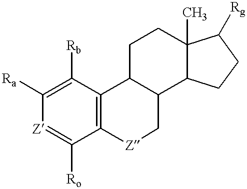

wherein the variables are defined in the specification.

### CROSS-REFERENCE TO RELATED APPLICATIONS

This application claims priority to U.S. Provisonal Patent Application No. 60/059,916 filed Sep. 24, 1997.

### BACKGROUND OF THE INVENTION

This invention relates to treating disease states characterized by abnormal cell mitosis.

Cell mitosis is a multi-step process that includes cell division and replication (Alberts, B. et al. In The Cell, pp. 652-661 (1989); Stryer, E. Biochemistry (1988)). Mitosis is characterized by the intracellular movement and segregation of organelles, including mitotic spindles and chromosomes. Organelle movement and segregation are facilitated by the polymerization of the cell protein tubulin. Microtubules are formed from α and β tubulin polymerization and the hydrolysis of GTP. Microtubule formation is important for cell mitosis, cell locomotion, and the movement of highly specialized cell structures such as cilia and flagella.

Microtubules are extremely labile structures that are sensitive to a variety of chemically unrelated anti-mitotic drugs. For example, colchicine and nocadazole are anti-mitotic drugs that bind tubulin and inhibit tubulin polymerization (Stryer, E. Biochemistry (1988)). When used alone or in combination with other therapeutic drugs, colchicine may be used to treat cancer (WO-9303729-A, published Mar, 4, 1993; J03240726-A, published Oct. 28, 1991), alter neuromuscular function, change blood pressure, increase sensitivity to compounds affecting sympathetic neuron function, depress respiration, and relieve gout (Physician's Desk Reference, Vol. 47, p. 1487, (1993)).

Estradiol and estradiol metabolites such as 2-methoxyestradiol have been reported to inhibit cell division (Seegers, J. C. et al. J. Steroid Biochem. 32, 797-809 (1989); Lottering, M-L. et al. Cancer Res. 52, 5926-5923 (1992); Spicer, L. J. and Hammond, J M. Mol. and Cell. Endo. 64, 119-126 (1989); Rao, P. N. and Engelberg, J. Exp. Cell Res. 48, 71-81 (1967)). However, the activity is variable and depends on a number of in vitro conditions. For example, estradiol inhibits cell division and tubulin polymerization in some in vitro settings (Spicer, L. J. and Hammond, J.M. Mol. and Cell. Endo. 64, 119-126 (1989); Ravindra, R., J. Indian Sci. 64(c) (1983)), but not in others (Lottering, M-L. et al. Cancer Res. 52, 5926-5923 (1992); Ravindra, R., J. Indian Sci. 64(c) (1983)). Estradiol metabolites such as 2-methoxyestradiol will inhibit cell division in selected in vitro settings depending on whether the cell culture additive phenol red is present and to what extent cells have been exposed to estrogen. (Seegers, J. C. et al. Joint NCI-IST Symposium. Biology and Therapy of Breast Cancer. 9125-9/27, 1989, Genoa, Italy, Abstract A58).

Numerous diseases are characterized by abnormal cell mitosis. For example, uncontrolled cell mitosis is a hallmark of cancer. In addition, cell mitosis is important for the normal development of the embryo, formation of the corpus luteum, wound healing, inflammatory and immune responses, angiogenesis and angiogenesis related diseases.

### SUMMARY OF THE INVENTION

The present invention provides compounds within the scope of the general formulae set forth below in the claims are useful for treating mammalian diseases characterized by undesired cell mitosis. Without wishing to be bound to any particular theory, such compounds generally inhibit microtuble formation and tubulin polymerization and/or depolymerization. Compounds within the general formulae having said inhibiting activity are preferred. Preferred compositions may also exhibit a change (increase or decrease) in estrogen receptor binding, improved absorbtion, transport (e.g. through blood-brain barrier and cellular membranes), biological stability, or decreased toxicity. The invention also provides compounds useful in the method, as described by the general formulae of the claims.

A mammalian disease characterized by undesirable cell mitosis, as defined herein, includes but is not limited to excessive or abnormal stimulation of endothelial cells (e.g., atherosclerosis), solid tumors and tumor metastasis, benign tumors, for example, hemangiomas, acoustic neuromas, neurofibromas, trachomas, and pyogenic granulomas, vascular malfunctions, abnormal wound healing, inflammatory and immune disorders, Bechet's disease, gout or gouty arthritis, abnormal angiogenesis accompanying: rheumatoid arthritis, psoriasis, diabetic retinopathy, and other ocular angiogenic diseases such as retinopathy of prematurity (retrolental fibroplasic), macular degeneration, corneal graft rejection, neovascular glaucoma and Osler Weber syndrome. Other undesired angiogenesis involves normal processes including ovulation and implantation of a blastula. Accordingly, the compositions described above can be used to block ovulation and implantation of a blastula or to block menstruation (induce amenorrhea).

The bond indicated by C...C is absent or, in combination with the C—C bond is the unit HC═CH.

Other features and advantages of the invention will be apparent from the following description of preferred embodiments thereof.

### BRIEF DESCRIPTION OF THE DRAWINGS

FIG. 1 is a graph illustrating the inhibition of tubulin polymerization by 2-methoxyestradiol described by Example 1 below.

FIG. 2 is a graph illustrating the inhibition of colchicine binding to tubulin by 2-methoxyestradiol described by Example 2 below.

FIG. 3 depicts: I. colchicine, 2-methoxyestradiol and combretastatin A-4, and II. various estradiol derivatives comprising colchicine (a-c) or combretastatin A-4 (d) structural motifs as described below.

### DETAILED DESCRIPTION OF THE INVENTION

As described below, compounds that are useful in accordance with the invention include novel estradiol derivatives that bind tubulin, inhibit microtubule formation or exhibit anti-mitotic properties. Specific compounds according to the invention are described below. Those skilled in the art will appreciate that the invention extends to other compounds within the formulae given in the claims below, having the described characteristics. These characteristics can be determined for each test compound using the assays detailed below and elsewhere in the literature.

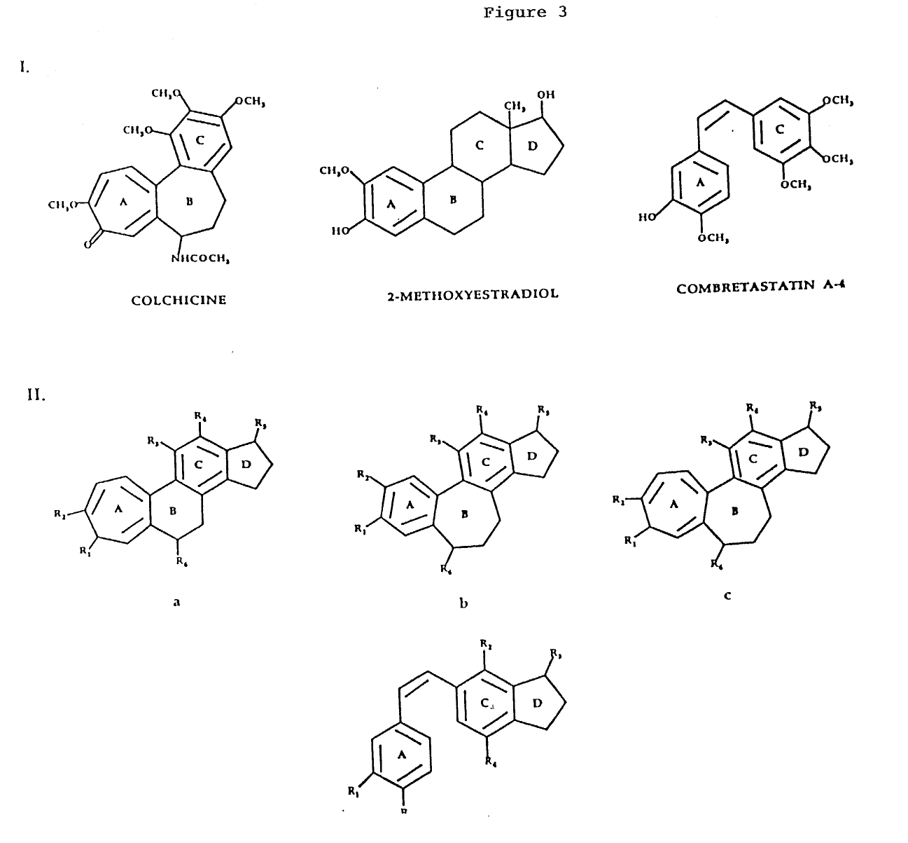

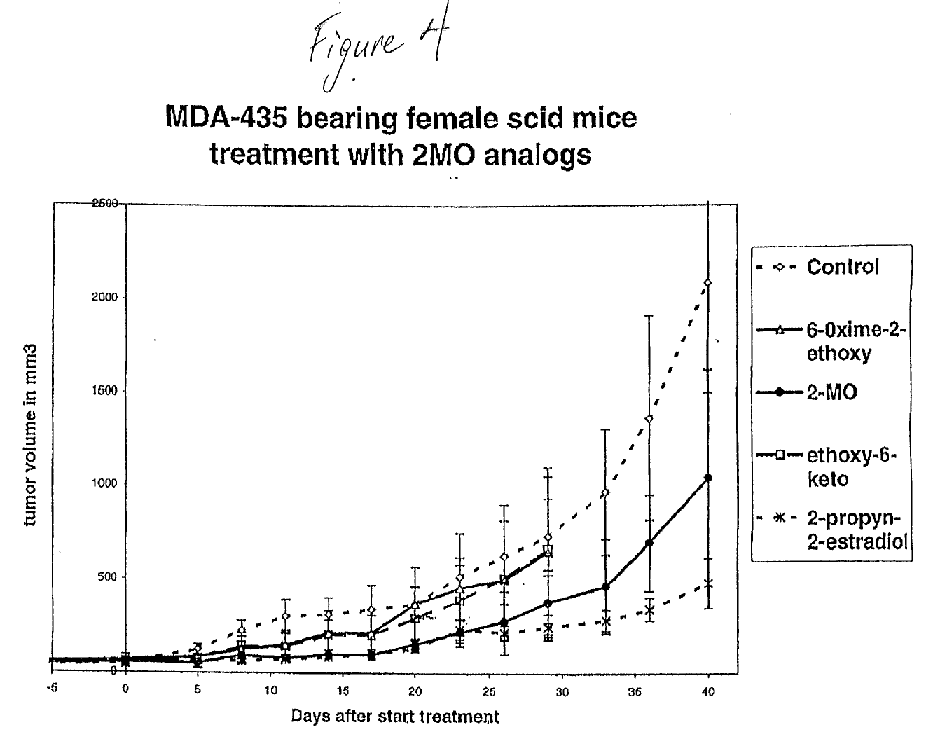

Without wishing to be bound to specific mechanisms or theory, it appears that certain compounds that are known to inhibit microtubule formation, bind tubulin and exhibit anti-mitotic properties such as colchicine and combretastatin A-4 share certain structural similarities with estradiol. FIG. 3 illustrates the molecular formulae of estradiol, colchicine, combretastatin A-4, and improved estradiol derivatives that bind tubulin inhibit microtubule assembly and exhibit anti-mitotic properties. Molecular formulae are drawn and oriented to emphasize structural similarities between the ring structures of colchicine, combretastatin A-4, estradiol, and certain estradiol derivatives. Estradiol derivatives are made by incorporating colchicine or combretastatin A-4 structural motifs into the steroidal backbone of estradiol.

FIG. 3, part I, depicts the chemical formulae of coichicine, 2-methoxyestradiol and combretastatin A-4. FIG. 3, part II a-d, illustrates estradiol derivatives that comprise structural motifs found in colchicine or combretastatin A-4. For example, part II a-c shows estradiol derivatives with an A and/or B ring expanded from six to seven carbons as found in colchicine and part IId depicts an estradiol derivative with a partial B ring as found in combretastatin A-4. Each C ring of an estradiol derivative, including those shown in FIG. 3, may be fully saturated as found in 2-methoxyestradiol. R₁₋₆ represent a subset of the substitution groups found in the claims. Each R₁-R₆ can independently be defined as -R₁, OR₁, -OCOR₁₁-SR₁, —F, —NHR₂, —Br, —I, or —C≡CH.

Anti-mitotic Activity In Situ

Anti-mitotic activity is evaluated in situ by testing the ability of an improved estradiol derivative to inhibit the proliferation of new blood vessel cells (angiogenesis). A suitable assay is the chick embryo chorioallantoic membrane (CAM) assay described by Crum et al. Science 230:1375 (1985). See also, U.S. Pat. No. 5,001,116, hereby incorporated by reference, which describes the CAM assay. Briefly, fertilized chick embryos are removed from their shell on day 3 or 4, and a methylcellulose disc containing the drug is implanted on the chorioallantoic membrane. The embryos are examined 48 hours later and, if a clear avascular zone appears around the methylcellulose disc, the diameter of that zone is measured. Using this assay, a 100 mg disk of the estradiol derivative 2-methoxyestradiol was found to inhibit cell mitosis and the growth of new blood vessels after 48 hours. This result indicates that the anti-mitotic action of 2-methoxyestradiol can inhibit cell mitosis and angiogenesis.

Anti-Mitotic Activity In Vitro

Anti-mitotic activity can be evaluated by testing the ability of an estradiol derivative to inhibit tubulin polymerization and microtubule assembly in vitro. Microtubule assembly is followed in a Gilford recording spectrophotometer (model 250 or 2400S) equipped with electronic temperature controllers. A reaction mixture (all concentrations refer to a final reaction volume of 0.25 μl) contains 1.0 M monosodium glutamate (ph 6.6), 1. omg/ml (10 μM) tubulin, 1.0 mM MgCl₂, 4% (v/v) dimethylsulfoxide and 20-75μM of a composition to be tested. The 0.24 ml reaction mixtures are incubated for 15 min. at 37° C. and then chilled on ice. After addition of 10p1 2.5mM GTP, the reaction mixture is transferred to a cuvette at 0° C., and a baseline established. At time zero, the temperature controller of the spectrophotometer is set at 37° C. Microtubule assembly is evaluated by increased turbity at 350 nm. Alternatively, inhibition of microtubule assembly can be followed by transmission electron microscopy as described in Example 2 below.

Indications

The invention can be used to treat any disease characterized by abnormal cell mitosis. Such diseases include, but are not limited to: abnormal stimulation of endothelial cells (e.g., atherosclerosis), solid tumors and tumor metastasis, benign tumors, for example, hemangiomas, acoustic neuromas, neurofribomas, trachomas, and pyogenic granulomas, vascular malfunctions, abnormal wound healing, inflammatory and immune disorders, Bechet's disease, gout or gouty arthritis, abnormal angiogenesis accompanying: rheumatoid arthritis, psoriasis, diabetic retinopathy, and other ocular angiogenic diseases such as retinopathy of prematurity (retrolental fibroplasic), macular degeneration, corneal graft rejection, neuroscular glacoma and Oster Webber syndrome.

In addition, the invention can be used to treat a variety of post-menapausal symptoms, including osteoporosis, cardiovascular disease, Alzheimer's disease, to reduce the incidence of strokes, and as an alternative to prior estrogen replacement therapies. The compounds of the present invention can work by estrogenic and non-estrogenic biochemical pathways.

Improved Estradiol Derivative Synthesis

Known compounds that are used in accordance with the invention and precursors to novel compounds according to the invention can be purchased, e.g., from Sigma Chemical Co., St. Louis, Steroloids and Research Plus. Other compounds according to the invention can be synthesized according to known methods from publicly available precursors.

The chemical synthesis of estradiol has been described (Eder, V. et al., Ber 109, 2948 (1976); Oppolzer, D. A. and Roberts, D A. Helv. Chim. Acta. 63, 1703, (1980)). Synthetic methods for making seven-membered rings in multi-cyclic compounds are known (Nakamuru, T. et al. Chem. Pharm. Bull. 10, 281 (1962); Sunagawa, G. et al. Chem. Pharm. Bull. 9, 81 (1961); Van Tamelen, E. E. et al. Tetrahedran 14, 8-34 (1961); Evans, D. E. et al. JACS 103, 5813 (1981)). Those skilled in the art will appreciate that the chemical synthesis of estradiol can be modified to include 7-membered rings by making appropriate changes to the starting materials, so that ring closure yields seven-membered rings. Estradiol or estradiol derivatives can be modified to include appropriate chemical side groups according to the invention by known chemical methods (The Merck Index, 11th Ed., Merck & Co., Inc., Rahway, N.J. USA (1989), pp. 583-584).

Administration

The compositions described above can be provided as physiologically acceptable formulations using known techniques, and these formulations can be administered by standard routes. In general, the combinations may be administered by the topical, oral, rectal or parenteral (e.g., intravenous, subcutaneous or intramuscular) route. In addition, the combinations may be incorporated into biodegradable polymers allowing for sustained release, the polymers being implanted in the vicinity of where delivery is desired, for example, at the site of a tumor. The biodegradable polymers and their use are described in detail in Brem et al., J. Neurosurg. 74:441-446 (1991). The dosage of the composition will depend on the condition being treated, the particular derivative used, and other clinical factors such as weight and condition of the patient and the route of administration of the compound. However, for oral administration to humans, a dosage of 0.01 to 100 mg/kg/day, preferably 0.01-1 mg/kg/day, is generally sufficient.

The formulations include those suitable for oral, rectal, nasal, topical (including buccal and sublingual), vaginal or parenteral (including subcutaneous, intramuscular, intravenous, intradermal, intraocular, intratracheal, and epidural) administration. The formulations may conveniently be presented in unit dosage form and may be prepared by conventional pharmaceutical techniques. Such techniques include the step of bringing into association the active ingredient and the pharmaceutical carrier(s) or excipient(s). In general, the formulations are prepared -by uniformly and intimately bringing into associate the active ingredient with liquid carriers or finely divided solid carriers or both, and then, if necessary, shaping the product.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil emulsion and as a bolus, etc.

A tablet may be made by compression or molding, optionally with one or more accessory ingredients. Compressed tablets may be prepared by compressing, in a suitable machine, the active ingredient in a free-flowing form such as a powder or granules, optionally mixed with a binder, lubricant, inert diluent, preservative, surface-active or dispersing agent. Molded tables may be made by molding, in a suitable machine, a mixture of the powdered compound moistened with an inert liquid diluent. The tablets may optionally coated or scored and may be formulated so as to provide a slow or controlled release of the active ingredient therein.

Formulations suitable for topical administration in the mouth include lozenges comprising the ingredients in a flavored basis, usually sucrose and acacia or tragacanth; pastilles comprising the active ingredient in an inert basis such as gelatin and glycerin, or sucrose and acacia; and mouthwashes comprising the ingredient to be administered in a suitable liquid carrier.

Formulations suitable for topical administration to the skin may be presented as ointments, creams, gels and pastes comprising the ingredient to be administered in a pharmaceutical acceptable carrier. A preferred topical delivery system is a transdermal patch containing the ingredient to be administered.

Formulations for rectal administration may be presented as a suppository with a suitable base comprising, for example, cocoa butter or a salicylate.

Formulations suitable for nasal administration, wherein the carrier is a solid, include a coarse powder having a particle size, for example, in the range of 20 to 500 microns which is administered in the manner in which snuff is taken, i.e., by rapid inhalation through the nasal passage from a container of the powder held close up to the nose. Suitable formulations, wherein the carrier is a liquid, for administration, as for example, a nasal spray or as nasal drops, include aqueous or oily solutions of the active ingredient.

Formulations suitable for vaginal administration may be presented as pessaries, tampons, creams, gels, pastes, foams or spray formulations containing in addition to the active ingredient such as carriers as are known in the art to be appropriate.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze-dried (lyophilized) conditions requiring only the addition of the sterile liquid carrier, for example, water for injections, immediately prior to use. Extemporaneous injection solutions and suspensions may be prepared from sterile powders, granules and tables of the kind previously described.

Preferred unit dosage formulations are those containing a daily dose or unit, daily sub-dose, as herein above recited, or an appropriate fraction thereof, of the administered ingredient.

It should be understood that in addition to the ingredients, particularly mentioned above, the formulations of this invention may include other agents convention in the art having regard to the type of formulation in question, for example, those suitable for oral administration may include flavoring agents.

### EXPERIMENTAL DATA

### EXAMPLE 1

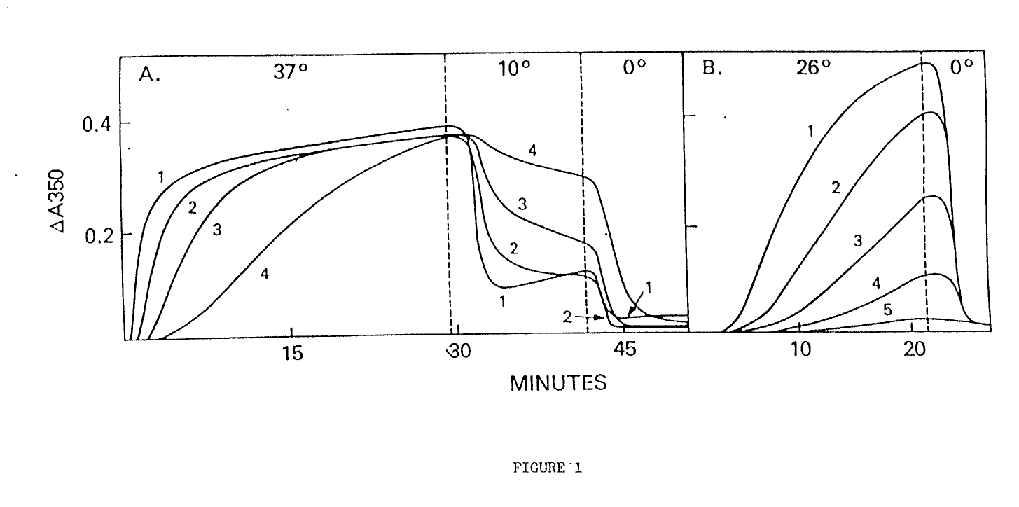

FIG. 1 illustrates the inhibition of tubulin polymerization by 2-methoxyestradiol.

A. Each reaction mixture (all concentrations refer to the final reaction volume of 0.25 ml) contained 1.0 M monosodium glutamate (pH 6.6), 1.0 mg/ml (10 μM) tubulin, 1.0 mM MgCl₂, 4% (v/v) dimethylsulfoxide, and either 0 (curve 1), 20μM (curve 2), 40 μM (curve 3), or 75 μM (curve 4) 2-methoxyestradiol. The 0.24 ml reaction mixtures were incubated for 15 min. at 37° C and chilled on ice. After addition of 10 μl of 2.5 mM GTP the reaction mixtures were transferred to cuvettes held at 0° C., and baselines were established. At time zero the temperature controller was set at 37° C. At the times indicated by the vertical dashed lines the temperature controller was set at the indicated temperatures.

B. Each reaction mixture contained 0.8 M monosodium glutamate (pH 6.6), 1.2 mg/ml (12 μM) tubulin, 4% (v/v) dimethylsulfoxide, and either 0 (curve 1), 1.0 FM (curve 2), 2.0 ,M (curve 3), 3.0 μM (curve 4), or 4.0 μM (curve 5) 2-methoxyestradiol. The 0.24 ml reaction mixtures were incubated for 15 min. at 26° C. and chilled on ice. After addition of 10μl of 10 mM GTP the reaction mixtures were transferred to cuvettes held at 0° C., and baselines were established. At time zero the temperature controller was set at 26° C. At the time indicated by vertical dashed line the temperature controller was set at 0° C.

### EXAMPLE 2

Transmission electron microscopy (TEM) can show differences between the morphology of polymerized tubulin formed in the absence or presence of 2-methoxyestradiol. After a 30 min. incubation (37° C.) of reaction mixtures containing the components described in Example 1, 75 μM 2-methoxyestradiol was added, and aliquots were placed on 200-mesh carbon coated copper grids and stained with 0.5% (w/v) uranyl acetate. TEM magnifications from 23,100× to 115,400× were used to visualize differences in tubulin morphology.

### EXAMPLE 3

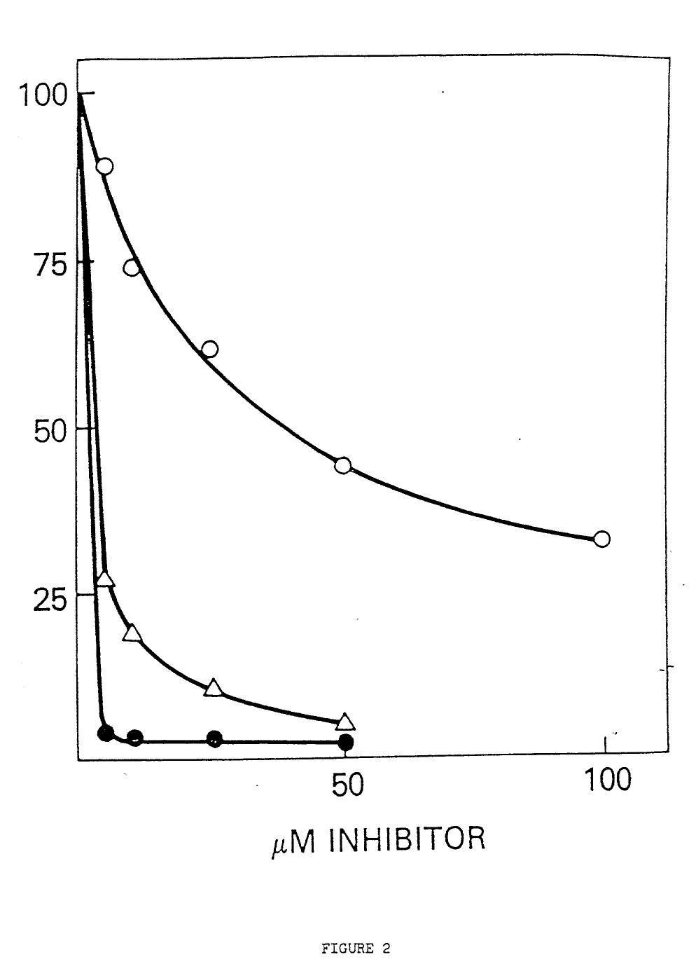

FIG. 2 illustrates that 2-methoxyestradiol inhibits colchicine binding to tubulin. Reaction conditions were as described in the text, with each reaction mixture containing 1.0 μM tubulin, 5% (v/v) dimethyl sulfoxide, 5 μM [³H]colchicine, and inhibitor at the indicated concentrations. Incubation was for 10 min. at 37° C. Symbols as follows: °, 2-methoxyestradiol, combretastatin A-4; Δ, dihydrocombretastatin A-4. Combretastatin A-4 and dihydrocombretastatin A-4 are compounds with anti-mitotic activity similar to colchicine.

### EXAMPLE 4

Table 1 illustrates the inhibitory effects on tubulin polymerization in vitro exhibited by estradiol or estradiol derivatives, plant anti-mitotic compounds such as colchicine, combretastatin A-4 or other plant compounds. The method is given in Example 1.

### EXAMPLE 5

Table 2 lists estrogens, estradiol or estradiol derivatives that inhibit colchicine binding to tubulin, by the method given in Example 3.

TABLE 1

| | IC₅₀ |
|---|---|
| | (μM ± S.D.) |
| ESTROGENIC COMPOUND | |
| 2-Methoxyestradiol | 1.9 ± 0.2 |
| Diethylstilbestrol | 2.4 ± 0.4 |
| 2-Bromoestradiol | 4.5 ± 0.6 |
| 2-Methoxyestrone | 8.8 ± 1 |
| 17-Ethynylestradiol | 10.0 ± 2 |
| 2-Fluoroestradiol | 27.0 ± 6 |
| Estradiol | 30.0 ± 6 |
| Estrone | >40 |
| 2-Methoxy-17-ethynylestradiol | >40 |
| Estriol | >40 |
| 2-Methoxyestriol | >40 |
| Estradiol-3-0-methyl ether | >40 |
| 2-Methoxyestradiol-3-0-methyl | >40 |
| ether | |
| 4-Methoxyestradiol | >40 |
| 4-Methoxyestradiol-3-0-methyl | >40 |
| ether | |
| Plant Products | |
| Colchicine | 0.80 ± 0.07 |
| Podophyllotoxin | 0.46 ± 0.02 |
| Combretastatin A-4 | 0.53 ± 0.05 |
| Dihydrocombretastatin A-4 | 0.63 ± 0.03 |

IC₅₀ values are defined as the concentration of an estradiol derivative required to inhibit tubulin polymerization by 50%. IC₅₀ values were obtained in at least two independent experiments for non-inhibitory agents (IC₅₀>40 μM) and at least three independent experiments for inhibitory compounds. IC₅₀ values were obtained graphically, and average values are presented. S.D., standard deviation.

TABLE 2

| | Percentage |
|---|---|
| ESTROGENIC COMPOUND | Inhibition |
| 2-Methoxyestradiol | 82 ± 2 |
| 2-Methoxyestrone | 57 ± 6 |
| 17-Ethynylestradiol | 50 ± 7 |
| Estradiol | 38 ± 4 |
| Diethylstilbestrol | 30 ± 4 |

Reaction conditions were described in Example 3, with each reaction mixture containing 1.0 μM tubulin, 5% (v/v) dimethyl sulfoxide, 2 μM [³H]colchicine, and 100 μM inhibitor. Incubation was for 10 mi. at 37° C. Average values obtained in three independent experiments are presented in the table, except for 2-methoxyestrone, which was only examined twice. S.D., standard deviation.

The following Examples refer to the compound of the general formula:

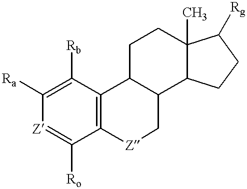

wherein:

a) R_{b} and Rₒ are independently —H, —Cl, —Br, —I, —F, —CN, lower alkyl, —OH, —CH₂—OH, —NH₂; or N(R₆)(R₇), wherein R₆ and R₇ are independently hydrogen or an alkyl or branched alkyl with up to 6 carbons;

b) Rₐ is —N₃,—C≡N, —N₃,—C C≡R, —C═CH-R, -R-C═CH₂, —C═CH, —O-R, -R-R₁, or —O-R-R₁ where R is a straight or branched alkyl with up to 10 carbons or aralkyl, and R₁ is —OH, —NH₂,—Cl, —Br, —I, —F or CF₃;

c) Z is >CH, >COH, or >C-R₂—OH, where R₂ is an alkyl or branched alkyl with up to 10 carbons or aralkyl;

d) >C-_{g} is >CH₂, >C(H)—OH, >C═O, >C═N—OH, >C(R₃)OH, >C═N—OR₃, >C(H)—NH₂, >C(H)—NHR₃, >C(H)—NR₃R₄, or >C(H)—C(O)-R₃, where each R₃ and R₄ is independently an alkyl or branched alkyl with up to 10 carbons or aralkyl; and

e) Z″ is >CH₂, >C═O, >C(H)—OH, >C═N—OH, >C═N—OR₅, >C(H)≡C═N, or >C(H)—NR₅R₅, wherein each R₅ is independently hydrogen, an alkyl or branched alkyl with up to 10 carbons or aralkyl.

### EXAMPLE 6

In Vitro Cellular Proliferation Inhibition Cells and Culture Conditions

MDA-MB-435 human breast carcinoma cells were grown in RPMI 1640 containing 10% heat-inactivated FCS (Hyclone Laboratories, Logan, Utah) and supplemented with 2 mM L-glutamine, 100 units/ml penicillin, 100 μg/ml streptomycin (Irvine Scientific, Santa Anna, Calif.). BCE cells were obtained as described previously (Folkman, J. Haudenschild, C. C., and Zetter, D. B., Long-term culture of capillary endothelial cells. Proc. Natl. Acad. Sci., USA, 76; 5217-5221, 1979) and grown on gelatinized surfaces [1.5 g/100 ml of gelatin in PBS (0.2 g/liter KCl, 0.2 g/liter KH₂PO₄, 8 g/liter NaCl, and 1.15 g/liter Na₂HPO4)] in DMEM containing 10% heat-inactivated bovine CS and supplemented with L-glutamine (2mm), penicillin (110 units/ml) and streptomycin (100 μg/ml) and 3 ng/ml bFGF. Both cell types were incubated at 37° C. under 10% CO₂ in air, RPMI 1640, DMEM, and CS were obtained from JRH Biosciences (Lenexa, Kans.).

Proliferation Assays

MDA-MB-435 cells were plated at 20,000 cells/ml in 24-well dishes. After allowing the cells to attach overnight, the appropriate fresh media were applied containing differing concentrations of 2-ME or derivatives thereof, as described below. Drug was made soluble in DMSO (Fisher Scientific, Pittsburgh, Pa.), and control wells received equal volumes (0.1%) of vehicle alone. Drug was added to the wells in a volume of 500 μl. The media for BCE cells was supplemented with bFGF (1 ng/ml), BCE cells were assayed in 5% CS, whereas MDA-MB-435 were assayed in 2.5% FCS because of their more rapid growth curves. The cells were incubated for 3 days at 37° C. and then washed with PBS, detached by trypsinization (0.05 g/100 ml trypsin, 0.53 mm EDTΔ, from Life Technologies, Inc., Grand Island, N.Y.), resuspended in Hematall (Fisher Scientific), and counted using a Coulter Counter. Each condition was prepared in triplicate, and the experiments were carried out three times. Results are presented as means ±SE.

Synthesis of 2-ME Derivatives

Synthesis of the 2-ME derivatives described herein is well within the capability of one ordinarily skilled in the art. A specific description of the synthesis of the 2-ME derivatives and analogs discussed herein can be found in M. Cushman, H-M. He, J. A. Katzenellenbogen, C. M. Lin and E. Hamel, Synthesis, antitubulin and antimitotic activity, and cytotoxicity of 2-methoxyestradiol, and endogenous mammalian metabolite of estradiol that inhibits tubulin polymerization by binding to the colchicine binding site, J. Med. Chem., 38(12): 2042 (1995); and M. Cushman, H-M. He, J. Katzenellenbogen, R. Varma, E. Hamel, C. Lin, S. Ram and Y. P. Sachdeva, Synthesis of analogs of 2-methoxyestradiol with enhanced inhibitory effects on tubulin polymerization and cancer cell growth, J. Med. Chem. 40(15): 2323 (1997).

Results

The tubulin activities, breast cancer cell line activities, and the cell panels most sensitive to selected analogs are shown in Tables 3 and 4. Some of the most potent analogs have also been examined for their binding affinities to the estrogen receptor. These results are also shown in Table 3.

2-ME and its analogs do not appear to be cytotoxic in vitro. Characteristically, 2-ME and the analogs all have molar log LC₅₀ =−4.0 in the cell panel assay, and several analogs which have been examined in vivo in mice have MTD>200 mg/kg.

The following structure is used for the compounds described in Table 3, where R_{b} and Rₒ are H, and >C-R_{g} is >C(H)-β-OH:

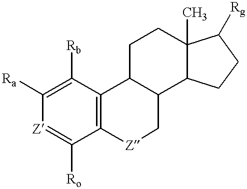

TABLE 3

| | | | | Tubulin | Tumor | |
|---|---|---|---|---|---|---|
| NSC | Ra | Z′ | Z″ | IC₅₀ (μM) | Panel* | RBA** |
| 659853 | OCH3 | C—OH | CH2 | 3.6 ± 0.4 | =6.63/−6.60 | 0.245 |
| 671043 | OC2H5 | C—OH | CH2 | 0.5 to 0.75 | −7.5/−7.7 | 0.011 |
| 678473 | OCH2CF3 | C—OH | CH2 | 1.7 | −6.10/−5.8 | |
| 667049 | CH═CH2 | C—OH | CH2 | 5.2 ± 1 | −5.6 | |
| 682429 | C≡C—CH3 | C—OH | CH2 | 2.2 | −6.13/−5.8 | |
| 667047 | E—CH═CHCH3 | C—OH | CH2 | 1.2 ± 0.1 | −7.0/−6.9 | |
| 673652 | NHC2H5 | C—OH | CH2 | ˜2.0 | −5.7/−5.7 | 0.35 |
| 673651 | NHCOCH3 | C—OH | CH2 | weak | −4.51 | |
| 679431 | OC2H5 | C—OH | C═O | 4.8 | −7.38/−6.6 | |
| 681684 | OCH2CF3 | C—OH | C═O | 6.0 | −5.06 | |
| 683688 | OC2H5 | C—OH | OH | 2.0 to 3.0 | −6.3 | |
| 680185 | OC2H5 | C—OH | C═NOH | 0.5 to 0.75 | −8/−7.6 | <0.001 |
| 681683 | OCH2CF3 | C—OH | C═NOH | 0.5 | −8/−7.6 | <0.0074 |
| 683125 | OC2H5 | C—OH | C═NOCH3 | 0.5 to 1.0 | −7.81/−7.3 | <0.001 |

*Average log IC₅₀(Molar) against 8 human breast cancer cell lines in the cell panel assay in vitro. Of these cell lines, only MCF-7 and T-47D express estrogen and progesterone receptors.
**Relative Binding Affinity to rat uterine cytosolic estrogen receptor at 0°. ³H-Estradiol═100.

TABLE 4
Most Sensitive Cell Lines in Human Tumor Cell Panel Assay
in vitro
(Most sensitive cell panes are marked with ✓)

| NSC | Breast* | CNS | Melanoma | Renal | Ovarian** |
|---|---|---|---|---|---|
| 659853 | ✓ | ✓ | ✓ | | ✓ |
| 671043 | ✓ | ✓ | | | ✓ |
| 667049 | ✓ | ✓ | ✓ | ✓ | ✓ |
| 682429 | ✓ | ✓ | | | ✓ |
| 667047 | ✓ | ✓ | ✓ | ✓ | ✓ |
| 679431 | ✓ | ✓ | ✓ | ✓ | ✓ |
| 683688 | ✓ | ✓ | ✓ | ✓ | ✓ |
| 680185 | ✓ | ✓ | ✓ | ✓ | ✓ |
| 681683 | ✓ | ✓ | ✓ | | ✓ |
| 683125 | ✓ | | ✓ | ✓ | |

*Of the 8 cell lines, only MCF-7 and T-47D express estrogen and progesterone receptors.
**Especially, OVCAR-3

### EXAMPLE 7

In Vivo Mouse Corneal Inhibition Mice

Five- to 7-week-old SCID female mice were obtained from Massachusetts General Hospital (Boston, Mass.), Immunocompetent 7-9-week-old female C57BL/6 mice were obtained from ARCH Technical Services, Children's Hospital (Boston, Mass.). All animal studies were conducted according to protocols approved by the Animal Ethics Committee of Children's Hospital. Animals were anesthetized in a methoxyflurane (Pittman-Moore, Mundelein, Ill.) chamber prior to all procedures and were observed until fully recovered. Animals were sacrificed by a lethal dose of methoxyflurane.

Mouse Corneal Micropocket Assay To study the effect of microtubule inhibitors on angiogenesis in vivo, a model of angiogenesis in the mouse cornea was used in C46BL/6 mice as described previously (Kenyon, B. B., Voest, E. E., Chen, C. C., Folkman, J., and D'Amato, R. J. A model of angiogenesis in the mouse cornea. Invest. Ophthalmol. & Visual Sci., 76: 1625-1632, 1996). In brief, after animals were anesthetized, corneal micropockets were created in both eyes with a modified von Graefe cataract knife. Into each pocket, a 0.4 mm ×0.4 mm×0.2 mm sucrose aluminum sulfate (Bukh Meditee, Copenhagen, Denmark) pellet coated with hydron polymer type NCC (IHN Sciences, New Brunswick, N.J.) containing 80 ng of bFGF or 160 ng of human recombinant FEGF (gift from Reprogenesis, Cambridge, Mass.) was implanted 1.0-1.2 mm from the limbal vessels (bFGF experiments) or 0.5-0.7 mm from the limbus (VEGF experiments). Erythromycin ointment (E. Fougera, Melville, N.Y.) was applied to each operated eye. The sucralfate acts to stabilize the growth factor and to slow its release from the hydron. Before testing each drug in this assay, we found the maximal dose that could be administered daily for 5 consecutive days without producing signs of toxicity (i.e., no hair loss, diarrhea, infection, lethargy, or weight loss). The treated groups received daily oral administration for 5 consecutive days of the stated dosage of 2-ME or a derivative thereof suspended in 0.5 g/100 ml of carboxymethylcellulose.

Treatment was started on the day of pellet implantation. Control mice received 0.1 ml of carboxymethylcellulose p.o. The vascular response (measured as the maximal vessel length and number of clock hours of neovascularization) was assessed on the fifth postoperative day, which was found to be the day of maximal angiogenic response. For this purpose, the eyes of the mice were examined by slit-lamp biomicroscopy. Area of corneal neovascularization was calculated using a modified formula for a half-ellipse: Area (mm²)=[π×clock hours×length (mm)×0.2 mm]. This formula provides the most accurate approximation of the area of neovascularization that grows toward the pellet. (Kenyon, B. B., Voest, E. E., Chen, C. C., Folkman, J., and D'Amato, R. J. A model of angiogenesis in the mouse cornea. Invest. Ophthalmol. & Visual Sci., 76: 1625-1632, 1996).

Results

The in vivo corneal pocket assay data provided that 2-ME (designated 659853) exhibited 25% inhibition when administered at a dosage of 150 mg/kg, the derivative designated 682429 exhibited 34% inhibition when administered at a dosage of 300 mg/kg, and the derivative designated 681684 exhibited 36% inhibition when administered at a dosage of 225 mg/kg. The derivative designated 681683 exhibited 22% inhibition when administered at a dosage of 150 mg/kg. The derivative designated 683125 exhibited 23% inhibition when administered at a dosage of 150 mg/kg.

### EXAMPLE 8

In Vivo Tumor Growth Inhibition

To determine the antitumor activity of 2-ME and derivatives, SCID mice were s.c. inoculated with 10⁶ MDA-MB-435 estrogen receptor-negative human breast carcinoma cells. Treatment was initiated on day 12 when tumor volumes reached 75-100 mm3 in volume. Drugs were suspended in 0.5 g/100 ml of carboxymethylcellulose with sterile glass beads, vortexed for 5 minutes, and administered p.o. in a volume of 0.1 ml. Control mice received 0.1 ml of carboxymethylcellulose p.o. Serial caliper measurements of perpendicular diameters were used to calculate tumor volume using the following: (shortest diameter)²×(longest diameter)×0.52. T/C was calculated as the volume of treated tumors over the volume of control tumors. A smaller number indicates a greater inhibition of tumor growth.

The results of the in vivo tumor growth-inhibition assay are shown in FIG. 4. The derivative 681683 (designated 6-oximine-2-ethoxy therein) performed approximately as well as the derivative 681684 (designated ethoxy-6-keto therein) in inhibiting the growth of the tumor. The most effective tumor inhibiting compound tested was derivative 682429 (designated 2-propyn-2-estradiol therein).

The in vivo assay data provided that 2-ME (designated 659853) had a T/C value of 0.4 to 0.5 when administered for 44 days at a dosage of 150 mg/kg (N=4), and the derivative designated 682429 had a T/C value of 0.25 when administered for 44 days at a dosage of 225 mg/kg (N=5, p<0.01 relative to controls for both). Further in vivo data provided that both 2-ME and the derivative designated 667047 had a T/C value of 0.40 when administered for 29 days at a dosage of 75 mg/kg (N=9, and p<0.01 relative to controls for both). Additional data indicated that the derivatives designated 671043, 679431, and 680185 each had a T/C value of 0.9 when administered at a dosage of 150 mg/kg.

All of the publications mentioned herein are hereby incorporated by reference in their entireties. The above examples are merely demonstrative of the present invention, and are not intended to limit the scope of the appended claims.

We claim:

1. A compound of the general formula: 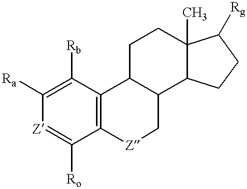
wherein:
a) R_{b} and Rₒ are independently —H, —Cl, —Br, —I, —F, —CN, lower alkyl, —OH, —CH₂—OH, —NH₂; or N(R₆)(R₇), wherein R₆ and R₇ are independently hydrogen or an alkyl or branched alkyl with up to 6 carbons;
b) Rₐ is —N₃,—C≡N, —C≡C-R, —C═CH-R, -R-C═CH₂, —C≡CH, —O-R, -R-R₁, or —O-R-R₁ where R is a straight or branched alkyl with up to 10 carbons or aralkyl, and R₁ is —OH, —NH₂, —Cl, —Br, —I, —F or CF₃;
c) Z′ is >CH, >COH, or >C-R₂—OH, where R₂ is an alkyl or branched alkyl with up to 10 carbons or aralkyl;
d) >C-R_{g} is >CH₂, >C(H)—OH, >C═O, >C═N—OH, >C(R₃)OH, >C═N-0R₃, >C(H)—NH₂, >C(H)—NHR₃, >C(H)—NR₃R₄, or >C(H)—C(O)-R₃, where each R₃ and R₄ is independently an alkyl or branched alkyl with up to 10 carbons or aralkyl; and
e) Z″ is >CH₂, >C═O, >C(H)—OH, >C═N—OH, >C═N—OR₅, >C(H) —C≡N, or >C(H)—NR₅R₅, wherein each R₅ is independently hydrogen, an alkyl or branched alkyl with up to 10 carbons or aralkyl.

2. The compound of claim 1, wherein:
R_{b} and Rₒ are H,
Rₐ is —C≡C —C—NH₃
Z′ is >C—OH,
>C-R_{g} is >C(H)-β-OH, and
Z″ is >CH₂.

3. The compound of claim 1, wherein:
R_{b} and Rₒ are H,
Rₐ is OCH₂CF₃
Z′ is >C—OH,
>C-R_{g} is >C(H)-β-OH, and
Z″ is >C═O.

4. The compound of claim 1, wherein:
R_{b} and Rₒ are H,
Rₐ is OCH₂CF₃
Z′ is >C—OH,
>C-R_{g} is >C(H)-β—OH, and
Z″ is >C═NOH.

5. The compound of claim 1, wherein:
R_{b} and Rₒ are H,
Rₐ is OC₂H₅
Z′ is >C—OH,
>C-R_{g} is >C(H)-β-OH, and
Z″ is >CH₂.

6. The compound of claim 1, wherein:
R_{b} and Rₒ are H,
Rₐ is OCH₂CF₃
Z′ is >C—OH,
>C-R_{g} is >C(H)-β-OH, and
Z″ is >CH₂.

7. The compound of claim 1, wherein:
R_{b} and Rₒ are H,
Rₐ is CH=CH₂
Z′ is >C—OH,
>C-R_{g} is >C(H)-β-OH, and
Z″ is >CH₂.

8. The compound of claim 1, wherein:
R_{b} and Rₒ are H,
Rₐ is E-CH=CHCH₃
Z′ is >C—OH,
>C-R_{g} is >C(H)-β-OH, and
Z″ is >CH₂.

9. The compound of claim 1, wherein:
R_{b} and Rₒ are H,
Rₐ is NHC₂H₅
Z′ is >C—OH,
>C-R_{g} is >C(H)-β-OH, and
Z″ is >CH₂.

10. The compound of claim 1, wherein:
R_{b} and Rₒ are H,
Rₐ is NHCOCH₃
Z′ is >C—OH,
>C-R_{g} is >C(H)-β-OH, and
Z″ is >CH₂.

11. The compound of claim 1, wherein:
R_{b} and Rₒ are H,
Rₐ is OC₂H₅
Z′ is >C—OH,
>C-R_{g} is >C(H)-β-OH, and
Z″ is >C═O.

12. The compound of claim 1, wherein:
R_{b} and Rₒ are H,
Rₐ is OC₂H₅
Z′ is >C—OH,
>C-R_{g} is >C(H)-β—OH, and
Z″ is >OH.

13. The compound of claim 1, wherein:
R_{b} and Rₒ are H,
Rₐ is OC₂H₅
Z′ is >C—OH,
>C-R_{g} is >C(H)-β—OH, and
Z″ is >C═NOH.

14. The compound of claim 1, wherein:
R_{b} and Rₒ are H,
Rₐ is OC₂H₅
Z′ is >C—OH,
>C-R_{g} is >C(H)-β-OH, and
Z″ is >C═NOCH₃.

15. A method of inhibiting angiogenesis comprising administering to an endothelial cell an angiogenesis inhibiting amount of a compound of the general formula: 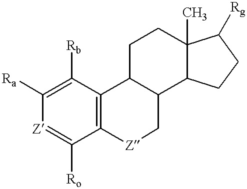
a) R_{b} and Rₒ are independently —H, —Cl, —Br, —I, —F, —CN, lower alkyl, —OH, —CH₂—OH, —NH₂; or N(R₆)(R₇), wherein R₆ and R₇ are independently hydrogen or an alkyl or branched alkyl with up to 6 carbons;
b)
Rₐ is —N₃, —CEN, —N₃, —C═---C≡R, —C═CH-R, -R-C═CH₂, —C≡CH, —O-R, -R-R₁, or —O-R-R₁ where R is a straight or branched alkyl with up to 10 carbons or aralkyl, and R₁ is —OH, —NH₂, —Cl, —Br, —I, —F or CF₃;
c)
Z′ is >CH , >COH, or
>C-R ₂—OH, where R₂ is an alkyl or bran ched alkyl with up to 10 carbons or aralkyl;
d)
>C-R_{g} is >CH₂, >C(H)—OH, >C═O, >C═N—OH, >C(R₃)OH, >C═N—OR₃, >C(H)—NH₂, >C(H)—NHR₃, >C(H)—NR-R₄, or >C(H)—C(O)-R₃, where each R₃ and R is independently an alkyl or branched alkyl with up to 10 carbons or aralkyl; and
e) Z″ is >CH₂, >C═O, >C(H)—OH, >C═N—OH, >C═N—OR₅, >C(H)—C≡N, or >C(H)—NR₅R₅, wherein each R₅ is independently hydrogen, an alkyl or branched alkyl with up to 10 carbons or aralkyl.

16. The method of claim 15, wherein:
R_{b} and Rₒ are H,
Rₐ is —C≡C —CH₃
Z′ is >C—OH,
>C-R_{g} is >C(H)-β-OH, and
Z″ is >CH₂.

17. The method of claim 15, wherein:
R_{b} and Rₒ are H,
Rₐ is OCH₂CF₃
Z′ is >C—OH,
>C-R_{g} is >C(H)-β-OH, and
Z″ is >C═O.

18. The method of claim 15, wherein:
R_{b} and Rₒ are H,
Rₐ is OCH₂CF₃
Z′ is >C—OH,
>C-R_{g} is >C(H)-β-OH, and
Z″ is >C═NOH.

19. The method of claim 15, wherein:
R_{b} and Rₒ are H,
Rₐ is OC₂H₅
Z′ is >C—OH,
>C-R_{g} is >C(H)-β-OH, and
Z″ is >CH₂.

20. The method of claim 15, wherein:
R_{b} and Rₒ are H,
Rₐ is OCH₂CF₃
Z′ is >C—OH,
>C-R_{g} is >C(H)-β-OH, and
Z″ is >CH₂.

21. The method of claim 15, wherein:
R_{b} and Rₒ are H,
Rₐ is CH=CH₂
Z′ is >C—OH,
>C-R_{g} is >C(H)-β-OH, and
Z″ is >CH₂.

22. The method of claim 15, wherein:
R_{b} and Rₒ are H,
Rₐ is E-CH=CHCH₃
Z′ is >C—OH,
>C-R_{g} is >C(H)-β—OH, and
Z″ is >CH₂.

23. The method of claim 15, wherein:
R_{b} and Rₒ are H,
Rₐ is NHC₂H₅
Z′ is >C—OH,
>C-R_{g} is >C(H)-β-OH, and
Z″ is >CH₂.

24. The method of claim 15, wherein:
R_{b} and Rₒ are H,
Rₐ is NHCOCH₃
Z′ is >C—OH,
>C-R_{g} is >C(H)-β-OH, and
Z″ is >CH₂.

25. The method of claim 15, wherein:
R_{b} and Rₒ are H,
Rₐ is OC₂H₅
Z′ is >C—OH,
>C-R_{g} is >C(H)-β-OH, and
Z″ is >C═O.

26. The method of claim 15, wherein:
R_{b} and Rₒ are H,
Rₐ is OC₂H₅
Z′ is >C—OH,
>C-R_{g} is >C(H)-β-OH, and
Z″ is >OH.

27. The method of claim 15, wherein:
R_{b} and Rₒ are H,
Rₐ is OC₂H₅
Z′ is >C—OH,
>C-R_{g} is >C(H)-β-OH, and
Z″ is >C═NOH.

28. The method of claim 15, wherein:
R_{b} and Rₒ are H,
Rₐ is OC₂H₅
Z′ is >C—OH,
>C-R_{g} is >C(H)-β—OH, and
Z″ is >C═NOCH₃.
